(19) United States
(12) Patent Application Publication
(10) Pub. No.: US 2017/0000511 A1
(43) Pub. Date: Jan. 5, 2017

(54) Surgical Device For Removing Tissue From Body

(71) Applicant: SAE WON MEDITECH.CO.,LTD., Bucheon-si (KR)

(72) Inventors: Sung-ho Maeng, Incheon (KR); Chang-young Kim, Bucheon-si (KR)

(21) Appl. No.: 14/361,233

(22) PCT Filed: May 19, 2014
(86) PCT No.: PCT/KR2014/004459

§ 371 Date: May 28, 2014

(30) Foreign Application Priority Data
May 19, 2014 (KR) 10-2014-00594

(51) Int. Cl. A61B 17/32

(52) U.S. Cl. CPC A61B 17/320068; A61B 2017/320008; A61B 17/56

(57) ABSTRACT

Provided is a casing; a controller that generates control signals; a vibration generating portion that is mounted in the casing and outputs vibration by receiving the control signals of the controller; vibration rods which extend to an outside of the casing in a state in which they are linked to the vibration generating portion, which are inserted into a body so that extending ends of the vibration rods reach a target point of the body, and which vibrate in their lengthwise directions according to an operation of the vibration generating portion; a scraper that closely contacts and is fixed to the extending ends of the vibration rods, vibrates in contact with an object to be removed and scrapes and removes the contacting object to be removed; and a bending unit that bends the scraper with a desired curvature and causes the scraper to closely contact the object to be removed.

### TECHNICAL FIELD

The present invention relates to a surgical device for removing a tissue from a body.

### BACKGROUND ART

In general, intervertebral discs that are usually called discs are placed between vertebrae that constitute the spine of a human body. The intervertebral discs play two roles of enabling movement of the spine in a state in which they are fixed between vertebral bodies that are adjacent in a vertical direction and of absorbing and alleviating shock.

Also, intervertebral foramina are formed between the vertebrae. The intervertebral foramina are passages through which neuromuscles diverged from spinal nerves pass. The neuromuscles pass through the intervertebral foramina and extend into each organ of the human body. In a normal case, the intervertebral foramina are wide enough for passage of the neuromuscles.

However, as the human body is aged, if bones that constitute the intervertebral foramina are snaggle-grown, if joints or ligaments are bloated, or if the intervertebral discs become thin, the intervertebral foramina become narrow. If the intervertebral foramina become narrow, the neuromuscles that pass through the intervertebral foramina are pressurized. As such, a patient may feel pain, or the patient's muscles may be weakened, and furthermore, the patient's walking becomes impossible, which may cause an obstacle in the patient's several body functions.

U.S. Patent Application Publication No. US200810161809 (published on Jul. 3, 2008)(hereinafter, referred to as ‘Prior Invention’) discloses an articulating tissue cutting device. The articulating tissue cutting device of the Prior Invention is a surgical instrument in which distal ends of the articulating tissue cutting device are inserted into intervertebral foramina and bloated tissues in the intervertebral foramina are cut using blades provided at the distal ends, thereby increasing the intervertebral foramina.

However, in the above-described Prior Invention, the blades operate manually using a trigger. For example, as the trigger is pulled once, the blades perform a cutting operation by making a reciprocal motion once. Thus, the speed of cutting is not fast, and it is very difficult for a surgeon to perform an operation using the blades as well as for the patient to bear the operation. Furthermore, since the surgeon has to grasp the trigger repeatedly by keeping a grip, a physical force is required to perform the operation.

Also, since the distal ends have a structure in which they are not flexible and are connected to a shaft portion using a pin and are bent, stress is concentrated on a connection portion, and the distal ends may be easily separated from the shaft portion. In particular, fine angle adjustment of the distal ends with respect to the shaft portion cannot be performed. The distal ends have to be in one state selected from a stretched state in a straight line and a bent state at a predetermined angle.

Furthermore, since the distal ends are thoroughly in the straight state, a surface to be cut cannot be covered with the distal ends such that a contact area is narrow and efficiency is lowered.

In addition, the direction of the distal ends cannot be precisely adjusted after the distal ends are inserted into the intervertebral foramina. In order to adjust the direction of the distal ends by inserting the distal ends into the intervertebral foramina, the surgeon has to rotate an actuating shaft portion and the shaft portion here and there by grasping a handle. In this case, other portions of the intervertebral foramina may be damaged.

### DETAILED DESCRIPTION OF THE INVENTION

### Technical Problem

The present invention provides a surgical device for removing a tissue from a body, whereby the surgical device has an active adjustment capability and thus can be smoothly inserted into intervertebral foramina and can be set in a correct position of the intervertebral foramina and a scraping plate for scraping an object to be removed is flexible and operates in a fully close contact with an affected area in the intervertebral foramina so that removing efficiency is excellent and the scraping plate is vibrated using a motor so that tissue removal can be finished within a very short time.

### Technical Solution

According to an aspect of the present invention, there is provided a surgical device for removing a tissue from a body, the surgical device including: a casing in which switches exposed to an outside are disposed; a controller that generates control signals by receiving signals from the switches; a vibration generating portion that is mounted in the casing and outputs vibration by receiving the control signals of the controller; vibration rods which extend to an outside of the casing in a state in which they are linked to the vibration generating portion, which are inserted into a body so that extending ends of the vibration rods reach a target point of the body, and which vibrate in their lengthwise directions according to an operation of the vibration generating portion; a scraper that closely contacts and is fixed to the extending ends of the vibration rods, vibrates in contact with an object to be removed and scrapes and removes the contacting object to be removed; and a bending unit that bends the scraper with a desire curvature and causes the scraper to closely contact the object to be removed.

A rod guide that is a hollow tube type member extending in a lengthwise direction, partially accommodate the vibration rods and guides guide motions of the vibration rods, may be further provided at the casing.

The vibration rods may include: a first vibration rod that is inserted into and extends to an inside of the rod guide in a state in which a rear end of the first vibration rod is fixed to the vibration generating portion; and a second vibration rod that is linked to an extending end of the first vibration rod inside the rod guide, extends to an outside of the rod guide and is flexible, and

The scraper that is an elastic piece having a predetermined thickness, may be fixed to the second vibration rod.

A flexible holder that supports the second vibration rod to be slidable in a lengthwise direction, may be fixed to the extending end of the rod guide.

The vibration generating portion may include: a motor mounted in the casing; a rotation member that is fixed to a driving shaft of the motor and rotates by driving of the motor; an eccentric pin that is fixed to the rotation member, is spaced apart from the driving shaft and is revolved around an axial line of the driving shaft when the rotation member rotates; and a vibration plate that is connected to a rear end of the first vibration rod, extends in a direction perpendicular to a motion direction of the first vibration rod and has a long hole in which the eccentric pin is accommodated.

The scraper may have a plurality of blades that are rectangular plate members formed of stainless steel, are capable of being detached from the second vibration rod and protrude in an opposite direction to the second vibration rod.

The bending unit may include: a trigger which is pivotably supported at the casing, of which part is placed in the casing and of which the remaining part is placed outside the casing; a traction member that is movably installed in the casing and is pulled in a direction far away from the rod guide when pulling the trigger; and a tensile wire that makes a front end of the flexible holder tensile when the traction member pulls the trigger by connecting the traction member and the front end of the flexible holder, thereby bending the flexible holder.

Two wire holes that are formed at a position in which they are eccentric from a central axis of the flexible holder and that are parallel to each other, may be formed in the flexible holder, and the tensile wire, of which one end is fixed to one side of the traction member, of which the other end passes through the rod guide and a one-side wire hole of the flexible holder and then which makes a U-turn, may pass through the opposite wire hole and an inside of the rod guide and may be fixed to the other side of the traction member.

The switches may include: an on/off switch that turns on/off the vibration generating portion; and a transmission switch that adjusts an output of the vibration generating portion.

### Effect of the Invention

A surgical device for removing a tissue from a body having the above configuration according to the present invention has an active adjustment unit and thus can be smoothly inserted into intervertebral foramina and can be set in a correct position of the intervertebral foramina and a scraping plate for scraping an object to be removed is flexible and operates in a fully close contact with an affected area in the intervertebral foramina so that removing efficiency is excellent and the scraping plate is vibrated using a motor so that tissue removal can be finished within a very short time.

### DESCRIPTION OF THE DRAWINGS

FIGS. 1 and 2 are perspective views of an external shape of a surgical device for removing a tissue from a body according to an embodiment of the present invention.

FIG. 3 is a view in a direction A of FIG. 1.

FIG. 4 is an exploded perspective view of part of the surgical device illustrated in FIG. 1.

FIGS. 5 through 7 are perspective views of an internal structure of the surgical device of FIG. 1.

FIG. 8 is an exploded view of a rod guide and a sliding holder illustrated in FIG. 7.

FIG. 9 is an exploded perspective view of a method of coupling a scraper to a second vibration rod illustrated in FIG. 8.

FIGS. 10 and 11 are views for explaining a bending principle of the scraper of the surgical device of FIG. 1.

### BEST MODE OF THE INVENTION

Hereinafter, a surgical device for removing a tissue from a body according to an embodiment of the present invention will be described with reference to the attached drawings in detail.

Basically, the surgical device for removing a tissue from a body according to the current embodiment of the present invention is an instrument that may be used for an orthopaedic operation. In particular, the surgical device for removing a tissue from a body has a structure that is suitable for removing joints or ligaments that protrude toward an inside of intervertebral foramina of the spine, or snaggle-grown bones. A tissue in a body that will be described later refers to a ligament or joint tissue that is grown or protrudes toward the inside of the intervertebral foramina or pressurizes neuromuscles, or a general tissue or bone.

The tissue in the body that is an induction factor that causes pain by pressurizing the neuromuscles that pass through a spinal canal, is an object to be removed and is easily removed using the surgical device for removing a tissue from a body according to the current embodiment.

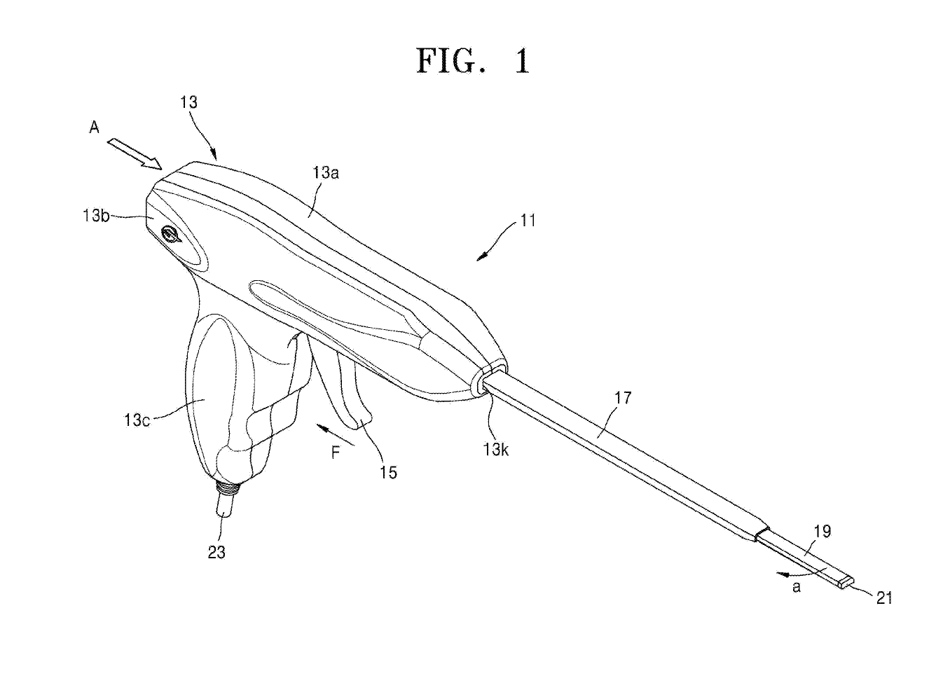

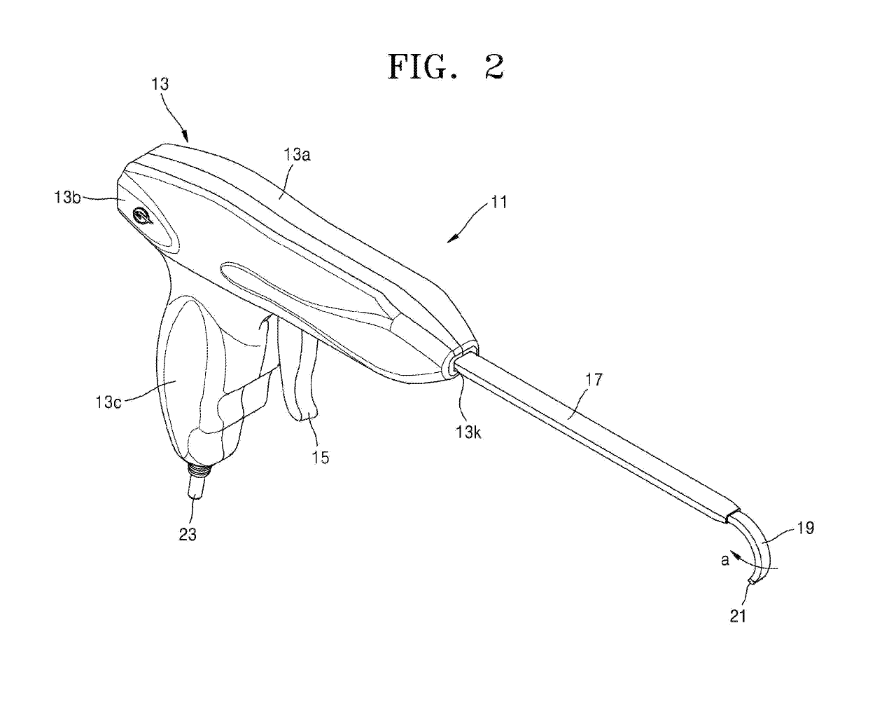

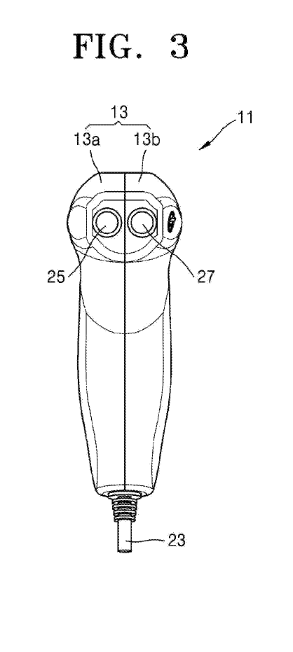

FIGS. 1 and 2 are perspective views of an external shape of a surgical device for removing a tissue from a body according to an embodiment of the present invention, and FIG. 3 is a view in a direction A of FIG. 1.

As illustrated in FIGS. 1 through 3, a surgical device 11 for removing a tissue from a body has a shape of a gun and includes a casing 13 having a handle portion 13c, a rod guide 17 that extends from a front end of the casing 13 in a lengthwise direction, a flexible holder 19 that is provided at the extending end of the rod guide 17 and may be bent, and a cap 21 that is fixed to a front end of the flexible holder 19.

Also, a trigger 15 is disposed in front of the handle portion 13c of the casing 13. The trigger 15 is a portion that is pulled by a user. If the trigger 15 is pulled and is pivoted in a direction of arrow F, the flexible holder 19 is bent in a direction of arrow a.

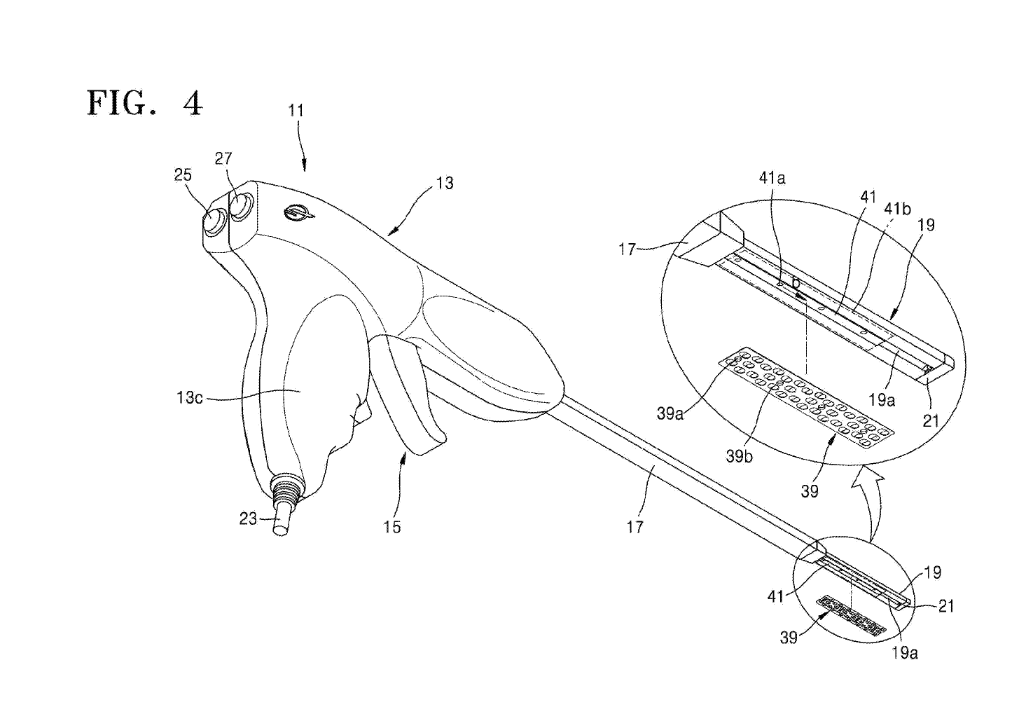

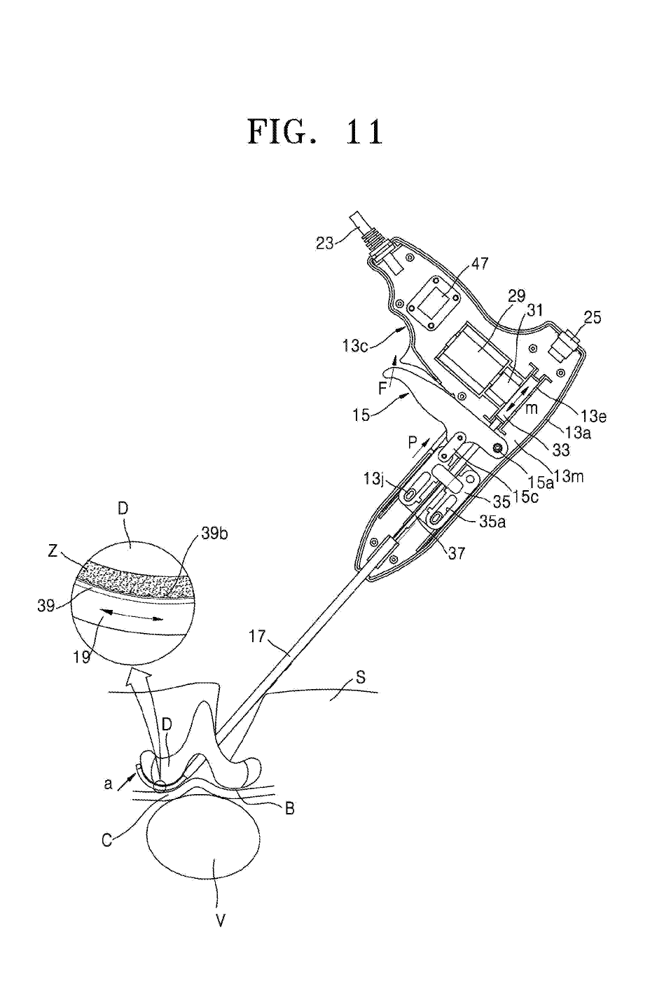

The flexible holder 19 is bent in the direction of arrow a so as to bend a scraper (39 of FIG. 4) disposed on a bottom surface of the flexible holder 19. Also, the scraper 39 is bent so as to be in widest contact with a surface to be removed, as illustrated in FIG. 11. A structure or an operating principle of the scraper 39 will be described later.

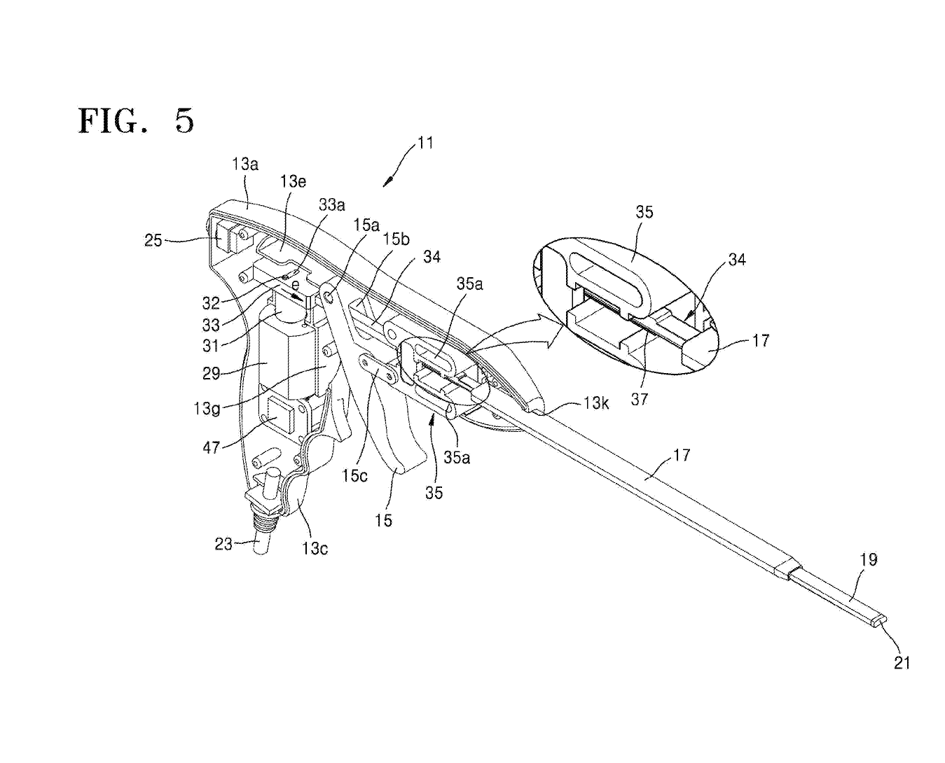

Reference numeral 23 is a power cable. The power cable 23 is an electric wire for supplying power to a motor (29 of FIG. 5) disposed in the casing 13.

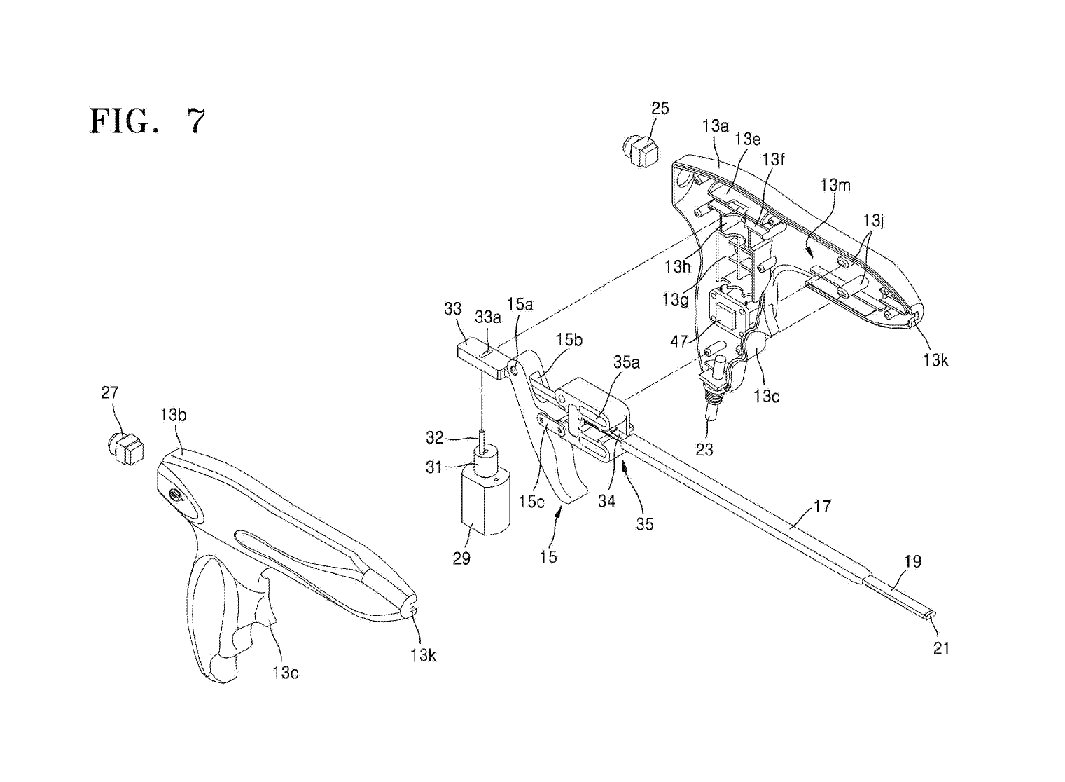

Also, the casing 13 includes two casing pieces 13a and 13b. The casing pieces 13a and 13b are symmetrical to each other and are assembled in a state in which the trigger 15 is inserted therebetween, thereby constituting the casing 13 that provides an internal space (13m of FIG. 7).

An on/off switch 25 and a transmission switch 27 are disposed in parallel at rear ends of the casing pieces 13a and 13b. The on/off switch 25 is a switch for turning on/off the motor 29, and the transmission switch 27 is a switch for adjusting the rotation speed of the motor 29.

The on/off switch 25 and the transmission switch 27 are connected to an inside of a controller (47 of FIG. 5) and cause the controller 47 to generate on/off signals or transmission signals (hereinafter, referred to as control signals). The controller 47 is electrically connected to the motor 29, generates the control signals when the on/off switch 25 or the transmission switch 27 is manipulated, and controls the motor 29.

The motor 29 may operate or may be stopped by manipulating the on/off switch 25, and revolutions per minute (rpm) of the motor 29 may be adjusted by the transmission switch 27. A switching method of the on/off switch 25 or the transmission switch 27 can be modified as long as it can perform these functions.

FIG. 4 is an exploded perspective view of part of the surgical device 11 illustrated in FIG. 1.

Referring to FIG. 4, the flexible holder 19 and a second vibration rod 41 extend to the front of the rod guide 17, and the scraper 39 is fixed to a bottom surface of the second vibration rod 41.

Since all of the flexible holder 19, the second vibration rod 41 and the scraper 39 have flexible properties, if the front end of the flexible holder 19 is pulled and is bent, as will be described later, the second vibration rod 41 and the scraper 39 are bent together.

The flexible holder 19 is a member that is fixed to a front end of the rod guide 17 and has a sliding groove 19a formed in a bottom surface of the flexible holder 19. The second vibration rod 41 is inserted into the sliding groove 19a. The second vibration rod 41 is slidable in a direction of arrow b in a state in which it is supported in the sliding groove 19a or in an opposite direction thereto.

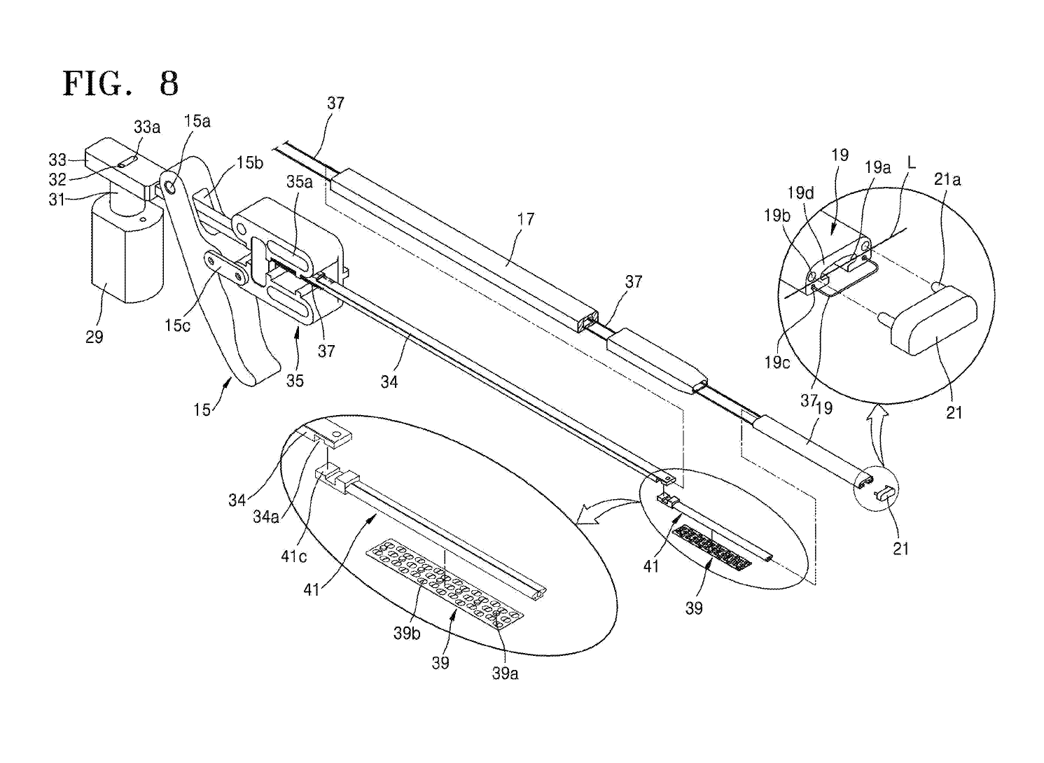

The second vibration rod 41 is a rod-shaped member, of which a rear end is connected to a front end of a first vibration rod (34 of FIG. 5) inside the rod guide 17, as illustrated in FIG. 8. The second vibration rod 41 is supported at the flexible holder 19 in a state in which it extends to the front of the rod guide 17.

Furthermore, a plurality of fixing grooves 41a are formed in the bottom surface of the second vibration rod 41. The plurality of fixing grooves 41a are grooves which one-to-one correspond to insertion protrusions 39a formed on the scraper 39 and in which the insertion protrusions 39a are accommodated and fixed.

When the insertion protrusions 39a are forcibly inserted into the fixing grooves 41a, the insertion protrusions 39a are coupled to the fixing grooves 41a so that the scraper 39 can be fixed to the second vibration rod 41. Reference numeral 41b is a position at which the scraper 39 is mounted on the second vibration rod 41.

The scraper 39 is manufactured by pressing a rectangular stainless steel plate having a predetermined thickness. The scraper 39 has a plurality of insertion protrusions 39a and a plurality of blades 39b. The plurality of insertion protrusions 39a are protrusions that are inserted into and coupled to the fixing grooves 41a of the second vibration rod 41.

Also, the plurality of blades 39b that protrude toward a bottom surface of the drawing, i.e., in an opposite direction to the flexible holder 19, scrapes an object to be removed that faces the blades 39b when the scraper 39 is vibrated. The shape of the blades 39b can be modified as long as they can perform this function.

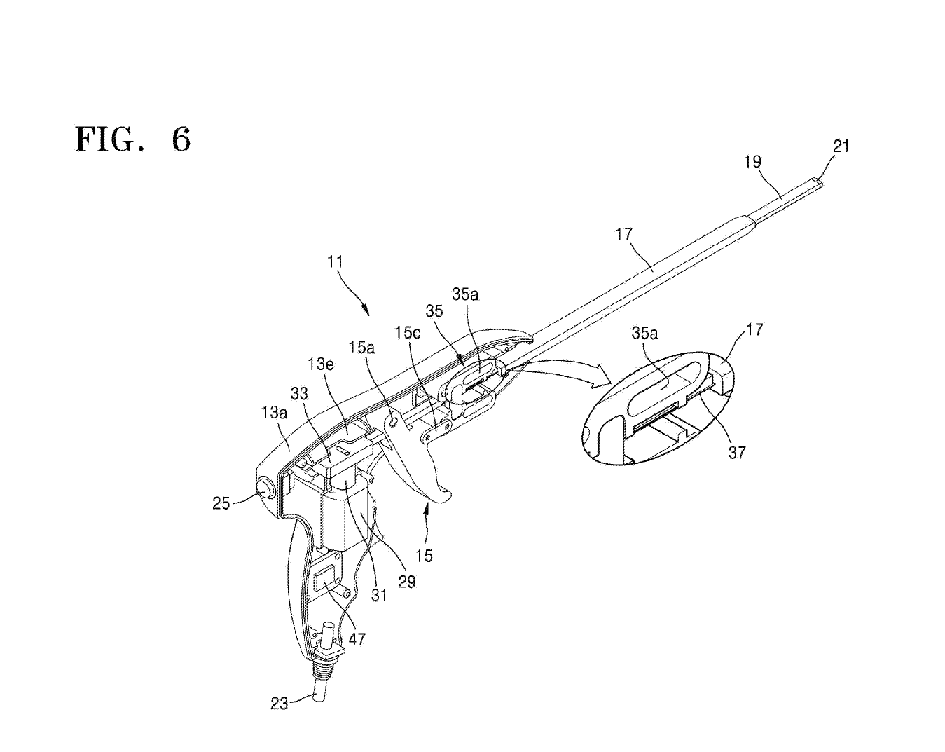

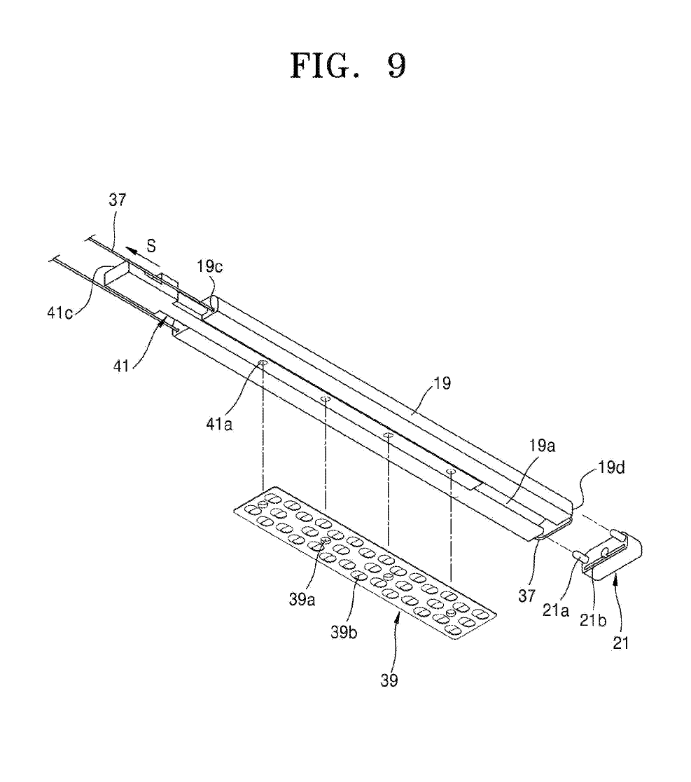

FIGS. 5 through 7 are perspective views of an internal structure of the surgical device 11 of FIG. 1. FIG. 8 is an exploded view of a rod guide and a sliding holder illustrated in FIG. 7, and FIG. 9 is an exploded perspective view of a method of coupling the scraper 39 to the second vibration rod 41 illustrated in FIG. 8.

As illustrated in FIGS. 5 through 9, a motor holding portion 13g, a rotation member accommodation groove 13h, and a vibration member guider 13e are integrally formed at inner sides of the casing pieces 13a and 13b. Although, in the drawings, only an internal shape of one-side casing piece 13a can be watched in terms of an angle, an internal shape of the opposite-side casing piece 13b is the same as that of the casing piece 13a.

The motor holding portion 13g is a support housing that accommodates and fixes the motor 29. When the casing pieces 13a and 13b are assembled by putting the motor 29 into the motor holding portion 13g, the motor 29 is fully fixed into the motor holding portion 13g.

Also, the rotation member accommodation groove 13h is a space in which a rotation member 31 that will be described later is rotatably accommodated, and the vibration member guider 13e supports a vibration plate 33 so that a vibration motion of the vibration plate 33 can be made in a direction of arrow e and in an opposite direction thereto.

Furthermore, the controller 47 is disposed below the motor holding portion 13g. The controller 47 outputs control signals by receiving manipulation signals of the switches 25 and 27 and transmits the control signals to the motor 29. The motor 29 is driven by the control signals generated by the controller 47. The motor 29 can be turned on/off and the rotation speed of the motor 29 can be controlled in response to the control signals of the controller 47.

As described above, the motor 29 operates in response to power signals transmitted from the controller 47 and has the rotation member 31 disposed at a driving shaft of the motor 29. The rotation member 31 that is a cylinder-shaped member having a predetermined diameter, axially-rotates when the motor 29 operates in a state in which the rotation member 31 is built in the rotation member accommodation groove 13h. A central axis of the rotation member 31 and the driving shaft of the motor 29 coincide with each other.

Also, an eccentric pin 32 is fixed to an upper part of the rotation member 31. The eccentric pin 32 is a member that is spaced apart from an axial line of the driving shaft of the motor 29 in parallel. When the motor 29 is driven, the eccentric pin 32 is revolved around the axial line of the driving shaft.

The vibration plate 33 that is a block-shaped member having a predetermined thickness, is slidably accommodated in an accommodation groove 13f of the vibration member guider 13e. In particular, a penetration long hole 33a is formed in the vibration plate 33. The penetration long hole 33a that is a hole extending in a direction perpendicular to a vibration direction of the vibration plate 33, accommodates the eccentric pin 32.

In this way, when the motor 29 operates in a state in which the eccentric pin 32 is inserted into the penetration long hole 33a, of course, the eccentric pin 32 causes the vibration plate 33 to vibrate in the direction of arrow e and in the opposite direction thereto.

The motor 29, the rotation member 31, the eccentric pin 32, and the vibration plate 33 serve as a vibration generating portion that outputs vibration by receiving the control signals of the controller 47.

As illustrated in FIG. 8, the first vibration rod 34 is fixed to the front of the vibration plate 33, and the second vibration rod 41 is connected to an extending end of the first vibration rod 34. The first and second vibration rods 34 and 41 form a straight line.

Furthermore, in order to connect the second vibration rod 41 to the first vibration rod 34 in a straight line, a hanging groove 34a is formed in the extending end of the first vibration rod 34, and a hanging protrusion 41c is formed at a rear end of the second vibration rod 41. When the hanging protrusion 41c is coupled into the hanging groove 34a, the second vibration rod 41 can be connected to the first vibration rod 34.

The first vibration rod 34 and the second vibration rod 41 are connected to an inside of the rod guide 17 and vibrate simultaneously with vibration of the vibration plate 33. The first and second vibration rods 34 and 41 vibrate in their lengthwise directions due to the motor 29.

Furthermore, the above-described scraper 39 is fixed to the bottom surface of the second vibration rod 41. The scraper 39 may be detached from the second vibration rod 41.

The rod guide 17 that is a hollow tube type member having a rectangular shape, is engaged in fixing holes 13k of the casing pieces 13a and 13b and partially accommodates the first and second vibration rods 34 and 41. Part of the first vibration rod 34 is built in the casing 13, and the other part of the first vibration rod 34 is built in the rod guide 17 and thus, the first vibration rod 34 is not exposed to the outside. Thus, the first vibration rod 34 does not directly contact a skin or tissue around the first vibration rod 34.

The rod guide 17 causes a combination of the flexible holder 19, the second vibration rod 41, and the scraper 39 to reach a target point of the body during a surgical procedure and supports the first and second vibration rods 34 and 41 to guide a vibration motion.

As the rod guide 17 supports the first and second vibration rods 34 and 41, the first and second vibration rods 34 and 41 slidably closely contact an inner wall surface of the rod guide 17. Thus, the first and second vibration rods 34 and 41 may vibrate only in a lengthwise direction of the rod guide 17.

The flexible holder 19 that is a member extending in its lengthwise direction while being fixed to the front end of the rod guide 17, has a sliding groove 19a formed in the center of a bottom surface of the flexible holder 19. Shapes of lengthwise cross-sections of the flexible holder 19 are the same.

The sliding groove 19a is a groove that accommodates the second vibration rod 41. The second vibration rod 41 (a portion of the second vibration rod 41 except for a portion having the hanging protrusion 41 formed therein) is slidable in its lengthwise direction in a state in which it is inserted into the sliding groove 19a. That is, the sliding groove 19a may vibrate in its lengthwise direction in a state in which it is supported at the flexible holder 19.

In particular, the sliding groove 19a is opened in a downward direction of the drawing so that the second vibration rod 41 can be coupled to the scraper 39.

Furthermore, insertion holes 19b and wire holes 19c are formed in both sides of the sliding groove 19a. The insertion holes 19b are holes into which insertion protrusions 21a formed at the cap 21 are inserted and coupled. The insertion protrusions 21a are forcibly inserted into the insertion holes 19b so that the cap 21 can be fixed to the flexible holder 19.

The wire holes 19c are penetration paths through which a tension wire 37 that will be described later passes, and extend in parallel along the lengthwise direction of the flexible holder 19. In particular, the wire holes 19c are eccentric from a widthwise center line L of the flexible holder 19.

In this way, the wire holes 19c are intended to be formed to be eccentric, because, when the tension wire 37 hung in the wire holes 19c is tensile in a direction of arrow s, even though the flexible holder 19 is pulled in a backward direction, the flexible holder 19 is bent in a direction of arrow a.

The tensile wire 37 will be described later.

The cap 21 is coupled to and fixed to a front end surface 19d of the flexible holder 19 and protects the tensile wire 37. The cap 21 has two insertion protrusions 21a and a wire accommodation groove 21b.

The two insertion protrusions 21a are protrusions that are inserted into and fixed into the insertion holes 19b of the flexible holder 19. Also, the wire accommodation groove 21b is a groove that accommodates part of the tensile wire 37 that is taken out from a one-side wire hole 19c and is inserted into the other side wire hole 19c across a widthwise direction, as enlarged and illustrated in FIG. 8. The wire accommodation groove 21b accommodates the tensile wire 37 at the front end of the flexible holder 19 so that the cap 21 can fully closely contact the front end surface 19d of the flexible holder 19.

The trigger 15, a traction member 35, and the tensile wire 37 are provided as a bending unit for bending the scraper 39 in the direction of arrow a of FIG. 1.

The trigger 15 is placed between the vibration generating portion and the rod guide 17, and a top end of the trigger 15 is supported in the casing 13 to be pivotable by a pivoting pin 15a. Furthermore, a top end of the trigger 15 extends to an outside of the casing 13 and is placed in front of the handle portion 13c.

Reference numeral 15b is a rod penetration hole through which the first vibration rod 34 passes. Since the rod penetration hole 15b is formed in this way, vibration of the vibration plate 33 can be transmitted to the first vibration rod 34 without attenuation in a state in which the first vibration rod 34 is not disturbed by the trigger 15.

The traction member 35 that is a member installed between the trigger 15 and the rod guide 17, is connected to a middle portion of the trigger 15 via a link member 15c and makes a rectilinear motion in a direction of arrow P of FIG. 11 when pulling the trigger 15. It is obvious that, when the trigger 15 is pivoted in an opposite direction, the traction member 35 is restored in an opposite direction to the direction of arrow P.

In order to make the rectilinear motion of the traction member 35, two guide long holes 35a are formed in upper and lower parts of the traction member 35, and traction member support pins 13j are disposed in the case 13. The traction member support pins 13j guide a horizontal motion of the traction member 35 in a state in which they are inserted into the guide long holes 35a.

The tensile wire 37 passes through both wire holes 19c in a state in which a central portion of the tensile wire 37 is hung in the front end surface 19d of the flexible holder 19, passes an inside of the rod guide 17, and both ends of the tensile wire 37 are fixed to the traction member 35, as illustrated in FIG. 9.

Thus, when the trigger 15 is pivoted in a direction of arrow F of FIG. 1 and pulls the traction member 35, the tensile wire 37 forcibly pulls the front end of the flexible holder 19. Since, as described above, the tensile wire 37 is hung at a position in which the tensile wire 37 is eccentric from the widthwise center line L of the flexible holder 19, the flexible holder 19 is bent in a downward direction due to a function of the tensile wire 37. As described above, as the flexible holder 19 is bent, the second vibration rod 41 and the scraper 39 are bent together.

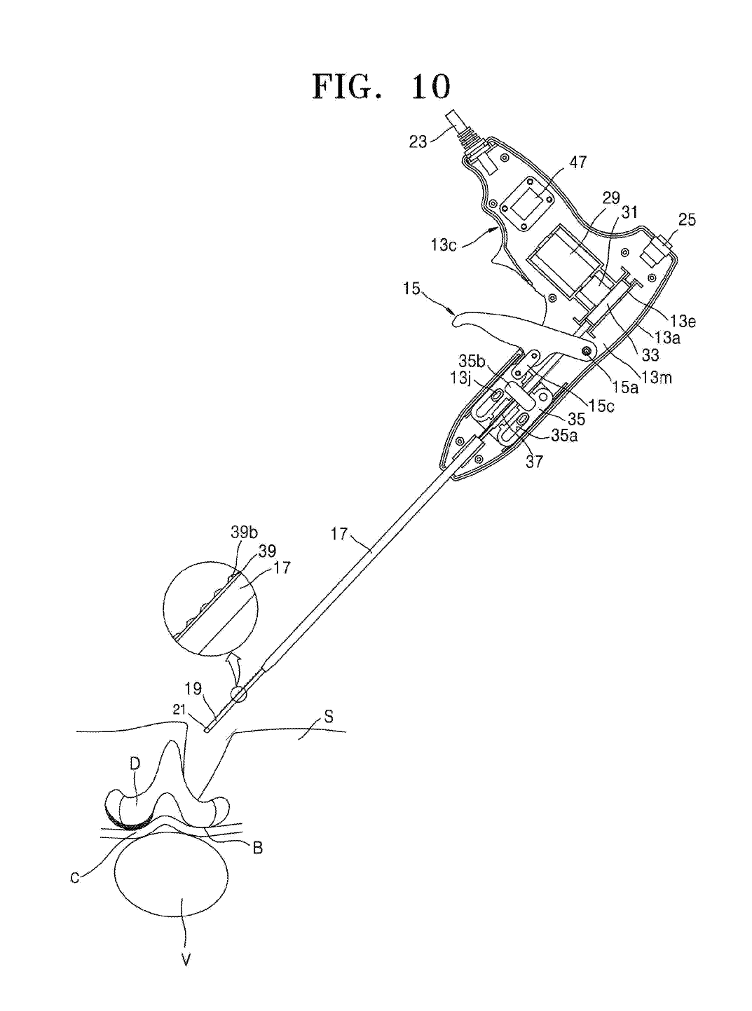

FIGS. 10 and 11 are views for explaining a bending principle of the scraper of the surgical device of FIG. 1.

Referring to FIG. 10, the flexible holder 19 is maintained in a straight line state due to its elasticity. Although the flexible holder 19, the second vibration rod 41 and the scraper 39 are flexible, they have their elasticity. Thus, they are maintained in the straight line state in a state in which no external force is applied thereto.

In order to remove an object Z to be removed in intervertebral foramina B of the spine in the above-described state, the rod guide 17 is inserted into the body via a cut skin S so that the scraper 39 reaches a target point of the intervertebral foramina B.

In this case, in order to closely contact the scraper 39 the object Z to be removed more widely and stably, the trigger 15 is pulled in the direction of arrow F, and the flexible holder 19 is bent as round as possible.

If the scraper 39 closely contacts the object Z to be removed through the procedure, the on/off switch 25 is manipulated so as to operate the motor 29. As the motor 29 operates, the vibration plate 33 vibrates in a direction of arrow m and a vibration force of the vibration plate 33 is transmitted to the scraper 39 via the first and second vibration rods 34 and 41.

The scraper 39 vibrates at a position in which the driving force of the motor 29 is transmitted to the scraper 39, and scrapes the object Z to be removed from an inferior articular process D. In this case, the vibration speed of the scraper 39 can be increased or decreased using the transmission switch 27 as needed.

If the object Z to be removed is sufficiently removed in the above-described procedure, the trigger 15 is put down, and the surgical device 11 for removing the tissue from the body is removed from the body so as to finish a surgical procedure, or the above-described procedure is repeatedly performed as needed.

Reference numeral C of FIG. 11 is neuromuscles pressurized by the object Z to be removed, and reference numeral V is a vertebral body.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A surgical device for removing a tissue from a body, the surgical device comprising:
a casing in which switches exposed to an outside are disposed;
a controller that generates control signals by receiving signals from the switches;
a vibration generating portion that is mounted in the casing and outputs vibration by receiving the control signals of the controller;
vibration rods which extend to an outside of the casing in a state in which they are linked to the vibration generating portion, which are inserted into a body so that extending ends of the vibration rods reach a target point of the body, and which vibrate in their lengthwise directions according to an operation of the vibration generating portion;
a scraper that closely contacts and is fixed to the extending ends of the vibration rods, vibrates in contact with an object to be removed and scrapes and removes the contacting object to be removed; and
a bending unit that bends the scraper with a desire curvature and causes the scraper to closely contact the object to be removed.

2. The surgical device of claim 1, wherein a rod guide that is a hollow tube type member extending in a lengthwise direction, partially accommodates the vibration rods and guides guide motions of the vibration rods, is further provided at the casing.

3. The surgical device of claim 2, wherein the vibration rods comprise:
a first vibration rod that is inserted into and extends to an inside of the rod guide in a state in which a rear end of the first vibration rod is fixed to the vibration generating portion; and
a second vibration rod that is linked to an extending end of the first vibration rod inside the rod guide, extends to an outside of the rod guide and is flexible, and
the scraper that is an elastic piece having a predetermined thickness, is fixed to the second vibration rod.

4. The surgical device of claim 3, wherein a flexible holder that supports the second vibration rod to be slidable in a lengthwise direction, is fixed to the extending end of the rod guide.

5. The surgical device of claim 3, wherein the vibration generating portion comprises:
a motor mounted in the casing;
a rotation member that is fixed to a driving shaft of the motor and rotates by driving of the motor;
an eccentric pin that is fixed to the rotation member, is spaced apart from the driving shaft and is revolved around an axial line of the driving shaft when the rotation member rotates; and
a vibration plate that is connected to a rear end of the first vibration rod, extends in a direction perpendicular to a motion direction of the first vibration rod and has a long hole in which the eccentric pin is accommodated.

6. The surgical device of claim 3, wherein the scraper has a plurality of blades that are rectangular plate members formed of stainless steel, are capable of being detached from the second vibration rod and protrude in an opposite direction to the second vibration rod.

7. The surgical device of claim 4, wherein the bending unit comprises:
a trigger which is pivotably supported at the casing, of which part is placed in the casing and of which the remaining part is placed outside the casing;
a fraction member that is movably installed in the casing and is pulled in a direction far away from the rod guide when pulling the trigger; and
a tensile wire that makes a front end of the flexible holder tensile when the traction member pulls the trigger by connecting the traction member and the front end of the flexible holder, thereby bending the flexible holder.

8. The surgical device of claim 7, wherein two wire holes that are formed at a position in which they are eccentric from a central axis of the flexible holder and that are parallel to each other, are formed in the flexible holder, and
the tensile wire, of which one end is fixed to one side of the traction member, of which the other end passes through the rod guide and a one-side wire hole of the flexible holder and then which makes a U-turn, passes through the opposite wire hole and an inside of the rod guide and is fixed to the other side of the traction member.

9. The surgical device of claim 1, wherein the switches comprise:
an on/off switch that turns on/off the vibration generating portion; and
a transmission switch that adjusts an output of the vibration generating portion.

10. A surgical device for removing a tissue from a body, the surgical device comprising:
a casing in which switches exposed to an outside are disposed;
a controller that generates control signals by receiving signals from the switches;
a vibration generating portion mounted in the casing and configured to output vibration by receiving the control signals from the controller;
vibration rods which extend to an outside of the casing and are linked to the vibration generating portion, the vibration rods being configured to be inserted into a body so that extending ends of the vibration rods reach a target point of the body and vibrate in a lengthwise direction when the vibration generating portion outputs the vibration;
a scraper that contacts and is fixed to the extending ends of the vibration rods, the scraper being configured to vibrate in contact with an object to be removed from the body and to scrape and remove the object; and
a bending unit configured to bend the scraper to a desired curvature and to cause the scraper to closely contact the object.

11. The surgical device of claim 10, further comprising a rod guide provided at the casing that is a hollow tube type member extending in a lengthwise direction, the guide rod partially accommodating the vibration rods and configured to guide motions of the vibration rods.

12. The surgical device of claim 11, wherein the vibration rods comprise:
a first vibration rod that is inserted into and extends to an inside of the rod guide, where a rear end of the first vibration rod is fixed to the vibration generating portion; and
a second vibration rod that is flexible and is linked to an extending end of the first vibration rod inside the rod guide, the second vibration rod extending to an outside of the rod guide, and
the scraper, the scraper being an elastic member having a predetermined thickness and being fixed to the second vibration rod.

13. The surgical device of claim 12, further comprising a flexible holder that is fixed to the extending end of the rod guide and supports the second vibration rod to be slidable in a lengthwise direction.

14. The surgical device of claim 12, wherein the vibration generating portion comprises:
a motor mounted in the casing;
a rotation member that is fixed to a driving shaft of the motor and that rotates by driving of the motor;
an eccentric pin that is fixed to the rotation member, spaced apart from the driving shaft and revolved around an axial line of the driving shaft when the rotation member rotates; and
a vibration plate that is connected to a rear end of the first vibration rod, extends in a direction perpendicular to a motion direction of the first vibration rod and has a long hole in which the eccentric pin is accommodated.

15. The surgical device of claim 12, wherein the scraper has a plurality of blades that are rectangular plate members formed of stainless steel, of the blades being detachable from the second vibration rod and protruding in an opposite direction to the second vibration rod.

16. The surgical device of claim 13, wherein the bending unit comprises:
a trigger pivotably supported at the casing, wherein a part of the trigger is placed in the casing and a remaining part of the trigger is placed outside the casing;
a fraction member movably installed in the casing, wherein the traction member is pulled in a direction away from the rod guide when pulling the trigger; and
a tensile wire connecting the traction member and a front end of the flexible holder and configured to cause the front end of the flexible holder to be tensile by bending the flexible holder when the traction member pulls the trigger.

17. The surgical device of claim 16, further comprising two parallel wire holes formed in the flexible holder at a position eccentric from a central axis of the flexible holder,
wherein one end of the tensile wire is fixed to one side of the traction member and another end of the tensile wire passes through the rod guide, passes through one of the two wire holes of the flexible holder, makes a U-turn, passes through another one of the two wire holes and an inside of the rod guide, and is fixed to another side of the traction member.

18. The surgical device of claim 1, wherein the switches comprise:
an on/off switch that turns on/off the vibration generating portion; and
a transmission switch that adjusts an output of the vibration generating portion.
